(19) United States
(12) Patent Application Publication
(10) Pub. No.: US 2025/0297227 A1
(43) Pub. Date: Sep. 25, 2025

(54) METHOD

(71) Applicants: AGENCY FOR SCIENCE, TECHNOLOGY AND RESEARCH, Singapore (SG); PANTHERA LABS PTE. LTD., Singapore (SG)

(72) Inventors: Kee Keong Adrian TEO, Singapore (SG); Lay Shuen TAN, Singapore (SG); Juin Ting CHEN, Singapore (SG); Kah Meng LIM, Singapore (SG)

(21) Appl. No.: 18/853,092

(22) PCT Filed: Mar. 7, 2023
(86) PCT No.: PCT/SG2023/050142

§ 371 Date: Sep. 30, 2024

(30) Foreign Application Priority Data
Mar. 31, 2022 (SG) 10202203309R

(51) Int. Cl. C12N 5/0775

(52) U.S. Cl. CPC C12N 5/0668; C12N 2500/32; C12N 2500/90; C12N 2501/115; C12N 2501/734; C12N 2506/45; C12N 2533/54

(57) ABSTRACT

The present invention relates generally to the field of stem cell differentiation. In particular, the invention relates to a serum-free method for differentiating stem cells into multipotent mesenchymal stromal cells (MSCs). The invention also relates to serum-free media for cell culture. In an aspect of the present invention, there is provided a serum-free composition for differentiating pluripotent stem cells into multipotent MSCs, the composition comprising Dulbecco's Modified Eagle Medium (DMEM), KnockOut™ Serum Replacement (KOSR) and GlutaMAX.

### CROSS-REFERENCE TO RELATED APPLICATIONS

The present application is a National Stage Application of International Application No. PCT/SG2023/050142, filed Mar. 7, 2023, which claims priority to and the benefit of Singapore patent application Ser. No. 10202203309R, filed Mar. 31, 2022, all of which are herein incorporated by reference in their entireties.

The present invention relates generally to the field of stem cell differentiation. In particular, the invention relates to a serum-free method for differentiating stem cells into multipotent mesenchymal stromal cells (MSCs). The invention also relates to serum-free media for cell culture.

Human MSCs are currently widely used in clinical trials for numerous cell applications. They are multipotent, safe (do not give rise to tumours) and are touted to have immuno-modulatory properties. However, primary human MSCs isolated from tissues are variable, heterogenous, and have limited expansion and proliferation capacities (˜20 passages).

Human pluripotent stem cells (hPSCs) are renewable and can provide an unlimited supply of MSCs for cell therapy and clinical trial applications. Unfortunately, current differentiation protocols to differentiate hPSCs into MSCs typically involve undefined reagents such as fetal bovine serum (FBS) and trypsin, which are not clinically-compliant.

There is thus a need for a method of generating MSCs in a sustainable and serum-free manner.

In one aspect of the present invention, there is provided a method of differentiating pluripotent stem cells into multipotent mesenchymal stromal cells (MSCs), the method comprising the steps of:
(a) culturing the stem cells in a first medium comprising Dulbecco's Modified Eagle Medium (DMEM), KnockOut™ Serum Replacement (KOSR) and GlutaMAX to form embryoid bodies (EBs);
(b) replacing the first medium with a second medium comprising DMEM, KOSR and GlutaMAX and culturing the EBs in the second medium to differentiate the EBs into MSC progenitors; and
(c) replacing the second medium with a third medium comprising DMEM, KOSR, GlutaMAX and Fibroblast Growth Factor 2 (FGF-2) and culturing the MSC progenitors in the third medium to differentiate the MSC progenitors into MSCs;
   wherein the first medium, the second medium and the third medium are serum-free.

As used herein, “serum-free” refers to the absence of animal or human serum. Serum-free compositions may include compositions that are free from fetal bovine serum or other bovine serum.

The term “differentiating” or “differentiation” as used herein refers to the developmental process by which a cell has progressed further down a developmental pathway than its immediate precursor cell. A differentiated cell is a cell of a more specialised cell type derived from a cell of a less specialised cell type in a cellular differentiation process. A differentiated cell is one that has taken on a more committed position within the lineage of the cell.

The term “stem cells” as used herein refers to cells capable of self-renewal and that are capable of differentiating into more specialised cells. As used herein, stem cells may include embryonic stem cells or induced pluripotent stem cells. The pluripotent stem cells as used herein may include but are not limited to human and non-human primate stem cells. Human pluripotent stem cells (hPSCs) may include human embryonic stem cells (hESCs) or human induced pluripotent stem cells (hiPSCs). In some embodiments, the pluripotent stem cells as used herein may be human embryonic stem cells, human induced pluripotent stem cells (hiPSCs), adult stem cells or primate induced pluripotent stem cells. Pluripotent stem cells have the potential to differentiate into tissues from all three embryonic germ layers (i.e. endoderm, mesoderm and ectoderm). Some of the tools for characterising pluripotency include quantitative PCR and immunofluorescence staining, which look for the upregulation of pluripotency genes such as Oct4, Sox2, and Nanog. However, the gold standard for evaluating pluripotency is a teratoma formation assay, which involves assessing the ability of cells to form tissues from all three germ layers in vivo in the form of an encapsulated tumour called a teratoma. By “embryoid bodies” (EBs), it is meant to include three-dimensional aggregates of pluripotent stem cells.

By “multipotent”, it is meant to include cell types that can give rise to a limited number of other specific cell types. Multipotent cells are committed to one or more embryonic cell fates, and thus, in contrast to pluripotent cells, cannot give rise to each of the three embryonic cell lineages as well as extraembryonic cells. Multipotent cells are more differentiated than pluripotent cells, but are not terminally differentiated.

The term “mesenchymal stromal cell” (MSC) as used herein refers to a multipotent cell that can be differentiated into cells of multiple lineages, such as chondrocytes, osteoblasts, adipocytes, and others. The term “MSCs” as used herein may refer to a population of cells in which 95% or more of the cells are MSCs, or in which 95% or more of the cells express MSC positive markers. MSC positive markers include CD73, CD90 and CD105. The term “MSCs” as used herein may refer to a population of cells defined according to the criteria proposed by the Mesenchymal and Tissue Stem Cell Committee of the International Society for Cellular Therapy (ISCT). One of the ISCT criteria is that MSCs are plastic-adherent when maintained in standard culture conditions. Another ISCT criteria is that ≥95% of the MSC population express CD105, CD73 and CD90. Additionally, MSCs lack expression (≤2% positive) of CD45, CD34, CD14 or CD11b, CD79a or CD19 and HLA-DR surface molecules. Another ISCT criteria is that MSCs are capable of differentiating in vitro to osteoblasts, adipocytes and chondroblasts. As used herein, a “progenitor MSC” or “MSC progenitor” refers to multipotent cells that have the potential to differentiate into MSCs. The term “MSC progenitors” as used herein may refer to a population of cells in which less than 95% of the cells express MSC positive markers.

In one embodiment, the DMEM is DMEM with a high glucose content, also known as “DMEM High Glucose”. A high glucose content in DMEM refers to a glucose content of 4.5 g/L (25 mmol/L) and a low glucose content in DMEM refers to a glucose content of 1 g/L (5.55 mmol/L). In some embodiments, the first medium, the second medium and the third medium contain DMEM High Glucose.

Alternatively, the DMEM may be DMEM/F-12 with GlutaMAX. DMEM/F-12 with GlutaMAX may be used in the dissociation of the pluripotent stem cells prior to step (a) of the method as described herein. DMEM/F-12 with GlutaMAX comprises a 1:1 mixture of DMEM (basal media version) and Ham's F-12. The glucose content of DMEM/F12 with GlutaMAX is 3.151 g/L.

The term “culturing” as used herein refers to growing a population of cells under suitable conditions for growth, in a liquid or solid medium. As used herein, the term “culturing” is meant to include subculturing, also known as passaging or cell splitting. Subculturing or passaging refers to a technique which keeps the cells alive and allows the cells to expand under culture conditions for extended periods of time. Subculturing or passaging may include the removal of the medium and transfer of cells from a previous culture into fresh growth medium to enable further expansion of the cells. The passaging procedure may also include the dissociation of cells prior to the transfer of cells into fresh growth medium.

In one embodiment, the method as described herein further comprises the step of dissociating the pluripotent stem cells into clumps using ReLeSR prior to step (a) or dissociating the pluripotent stem cells into single cells using TrypLE, prior to step (a). The use of ReLeSR or TrypLE instead of trypsin for the dissociation of the pluripotent stem cells is advantageous for the reasons set out below. As trypsin is porcine-derived, it is xenogenic and unsuited for cell therapy applications. On the other hand, TrypLE is animal origin-free and ReLeSR is cGMP qualified.

Compared to trypsin, TrypLE is gentler on cells and may be able to preserve surface epitopes such as CD73, CD90 and CD105. While trypsin requires neutralisation with serum-containing buffers/media, which posits a problem for cell therapy manufacturing, the enzymatic activity of TrypLE can be inhibited by dilution of TrypLE, allowing for serum-free reagents to be used during the passaging of cells.

The inventors also found ReLeSR to be superior to trypsin as, depending on the duration of ReLeSR treatment, ReLeSR has the differential ability to allow for the detachment of stem cells, whilst allowing for differentiated cells to remain attached on the cell plate. Based on the experiments carried out, the inventors found that trypsin indiscriminately detaches all (differentiated and undifferentiated) cells. Therefore, the use of ReLeSR in the dissociation step gives better assurance that clumps subsequently formed are from pluripotent stem cells.

In one embodiment, prior to the dissociation step, the pluripotent stem cells are cultured in MSC M0 medium shown in Table 1 or mTeSR™ 1 (Stemcell Technologies, catalogue number 85850). During the dissociation of the pluripotent stem cells into clumps using ReLeSR or into single cells using TrypLE for EB formation, the pluripotent stem cells are cultured in hPSC M0 medium shown in Table 1 or DPBS (Cytiva, catalogue number SH30028) to dilute the dissociation reagents (i.e. ReLeSR or TrypLE) and stop the reaction.

In one embodiment, the pluripotent stem cells dissociated using ReLeSR are cultured in MSC M1 medium for EB formation. In another embodiment, the pluripotent stem cells dissociated using TrypLE are cultured with MSC M0 medium and 5-10 μM of Y-27632 for 24 hours and subsequently with MSC M1 medium for EB formation. In one embodiment, EBs are formed 1-2 days in culture after the dissociation of the pluripotent stem cells.

In one embodiment, the EBs and MSC progenitors are cultured on a gelatin-coated plate. The use of a gelatin coating provides an ideal cell-attachment substrate for cell culture and may delay senescence of the cells.

In one embodiment, the MSC progenitors are dissociated with TrypLE prior to step (c) of the method as described herein.

In one embodiment of step (b) of the method as described herein, the differentiation of EBs to MSCs begins upon the replating of EBs onto gelatin-coated plates and culturing with MSC M2 medium. In one embodiment, the replating is done at Day 8. At this stage, the cells begin to take on MSC-like morphology but are classified as MSC progenitors rather than MSCs as the majority of the cells have yet to express MSC cell markers such as CD73, CD90 and CD105. In one embodiment, the replacement of MSC M2 medium with MSC M3 medium in step (c) represents passage 1. The cells are passaged using MSC M3 medium. During the first few passages, the cells may still be MSC progenitors as the majority of the cells have yet to express MSC cell markers such as CD73, CD90 and CD105. After the first few passages, a high purity MSC population (with 95% or more of the cells expressing MSC positive markers and less than 2% of the cells expressing MSC negative markers such as CD34 and CD45) is obtained and the cells are then classified as MSCs. In one embodiment, MSCs are formed after passage 5.

In one embodiment, the method further comprises passaging the MSCs obtained in step (c) in the third medium to maintain the MSCs in a multipotent state.

In a further embodiment, the MSCs are maintained in a multipotent state after 15 passages using the third medium.

In another embodiment, the MSCs are dissociated with TrypLE prior to each passage using the third medium.

By “maintaining”, it is meant to include processes that keep the cells viable. As used herein, the term “maintaining” in the context of maintaining cells in a multipotent state may include the passaging of the cells to keep the cells at an optimal density for continued growth and to preserve the multipotency of the cells.

In one embodiment, the first medium is MSC M1 Medium, the second medium is MSC M2 Medium, and the third medium is MSC M3 Medium shown in Table 1.

MSC M3 medium may also be a MSC maintenance medium to maintain the MSCs in a multipotent state. The presence of FGF-2 in MSC M3 medium may contribute to maintaining the multipotency of the MSCs.

In one embodiment, the culturing of the pluripotent stem cells to form EBs in the first medium, the culturing of the EBs to form MSC progenitors in the second medium, the culturing of the MSC progenitors to form MSCs in the third medium, and the maintenance of MSCs in the third medium are carried out at a temperature of 37° C., CO₂ level of 5%, and pH of 7.4.

In one embodiment, the pluripotent stem cells in step (a) of the method as described herein are human induced pluripotent stem cells (hiPSCs).

In one embodiment of the method as described herein, after step (c), at least 95% of the population of cells obtained are MSCs.

In another aspect of the present invention, there is provided a serum-free composition for differentiating pluripotent stem cells into multipotent MSCs, the composition comprising Dulbecco's Modified Eagle Medium (DMEM), KnockOut™ Serum Replacement (KOSR) and GlutaMAX.

In one embodiment of the serum-free composition as described herein, DMEM is present at a concentration of about 74% to about 89% (v/v), KOSR is present at a concentration of about 10% to about 25% (v/v), and GlutaMAX is present at a concentration of about 1% (v/V).

In one embodiment of the serum-free composition as described herein, DMEM is present at a concentration of about 79% to about 84% (v/v), KOSR is present at a concentration of about 15% to about 20% (v/v) and GlutaMAX is present at a concentration of about 1% (v/V).

In yet another embodiment of the serum-free composition as described herein, DMEM is present at a concentration of about 79% to about 89% (v/v), KOSR is present at a concentration of about 10% to about 20% (v/v) and GlutaMAX is present at a concentration of about 1% (v/v).

In one embodiment, the serum-free composition further comprises Fibroblast Growth Factor 2 (FGF-2).

In another embodiment of the serum-free composition, DMEM is present at a concentration of about 74% to about 84% (v/v), KOSR is present at a concentration of about 15% to about 25% (v/v), GlutaMAX is present at a concentration of about 1% (v/v) and FGF-2 is present at a concentration of about 2.5 ng/ml to about 5 ng/ml. In another embodiment, FGF-2 is present at a concentration of about 2.5 ng/ml.

In one embodiment, the pluripotent stem cells are human induced pluripotent stem cells (hiPSCs).

In another aspect of the invention, there is provided a population of MSCs obtained by or obtainable by the method as described herein.

In one embodiment, at least 95% of cells in the population of MSCs express CD73, CD90 and CD105.

In one embodiment, 2% or less of cells in the population of MSCs express CD34 and CD45.

The listing or discussion of an apparently prior-published document in this specification should not necessarily be taken as an acknowledgement that the document is part of the state of the art or is common general knowledge.

Any document referred to herein is hereby incorporated by reference in its entirety.

In order that the present invention may be fully understood and readily put into practical effect, there shall now be described by way of non-limitative examples only preferred embodiments of the present invention, the description being with reference to the accompanying illustrative figures.

In the Figures:

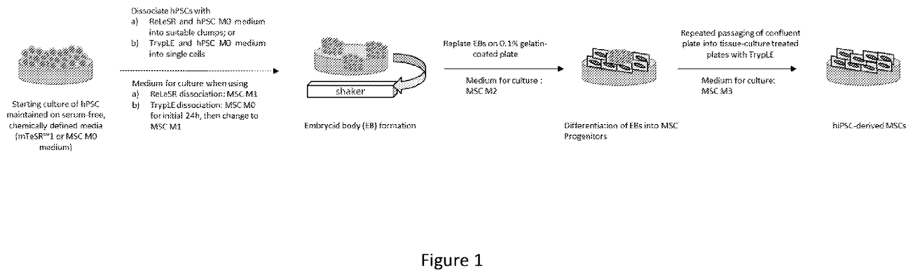

FIG. 1 is schematic illustration of the clinically-compliant serum-free protocol to generate hPSC-derived MSCs from starting hPSC culture.

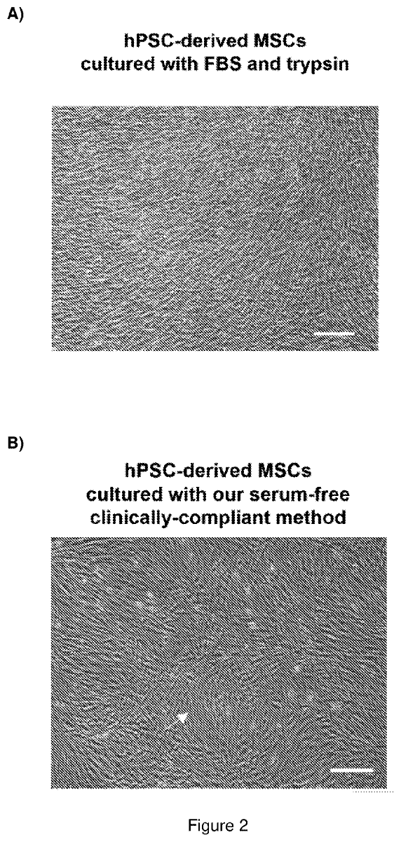

FIG. 2 shows representative bright field images of hPSC-derived MSCs generated with different methods. A) shows hPSC-derived MSCs generated with FBS and trypsin. B) shows hPSC-derived MSCs generated with serum-free defined alternatives-KOSR and TrypLE. Scale bar represents 10 μm.

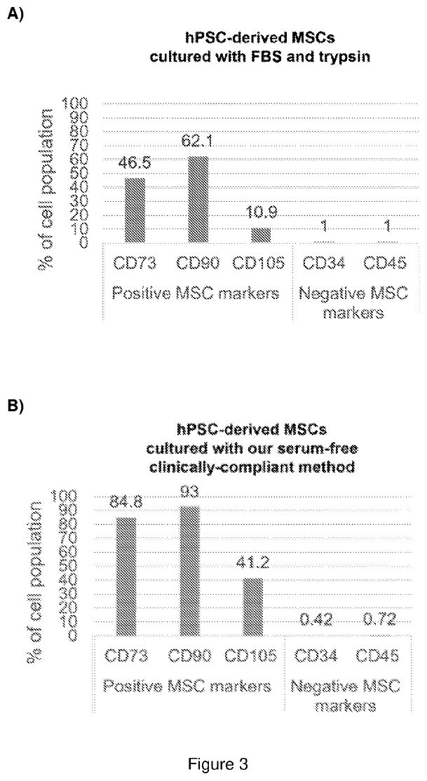

FIG. 3 shows flow cytometry analysis of MSC population that was generated with different methods. A) shows hPSC-derived MSCs generated with FBS and trypsin. B) shows hPSC-derived MSCs generated with serum-free defined alternatives-KOSR and TrypLE. MSC populations used were cultured over the same period of time.

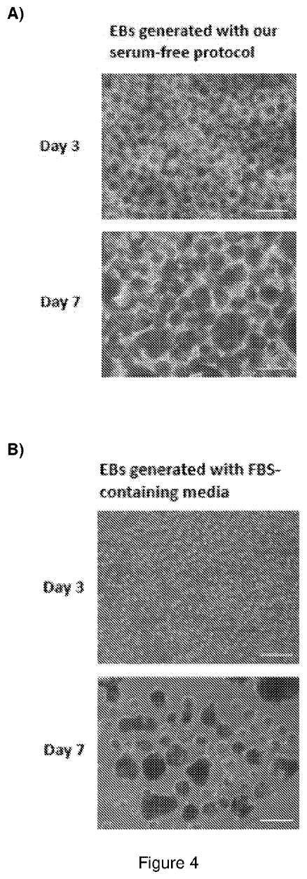

FIG. 4 shows representative brightfield images of EBs generated with different methods over seven days in culture. EBs generated with A) serum-free defined alternative-KOSR and B) FBS. Scale bar represents 10 μm.

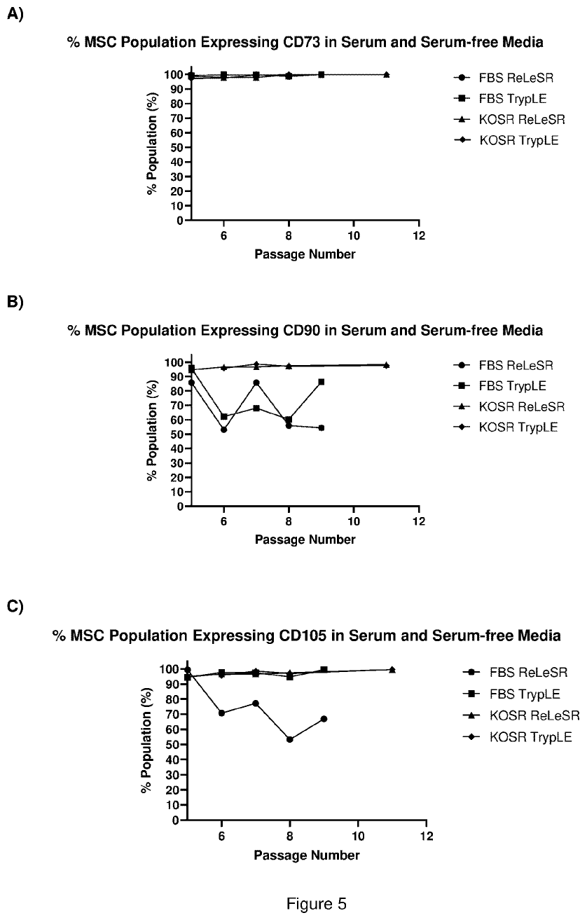

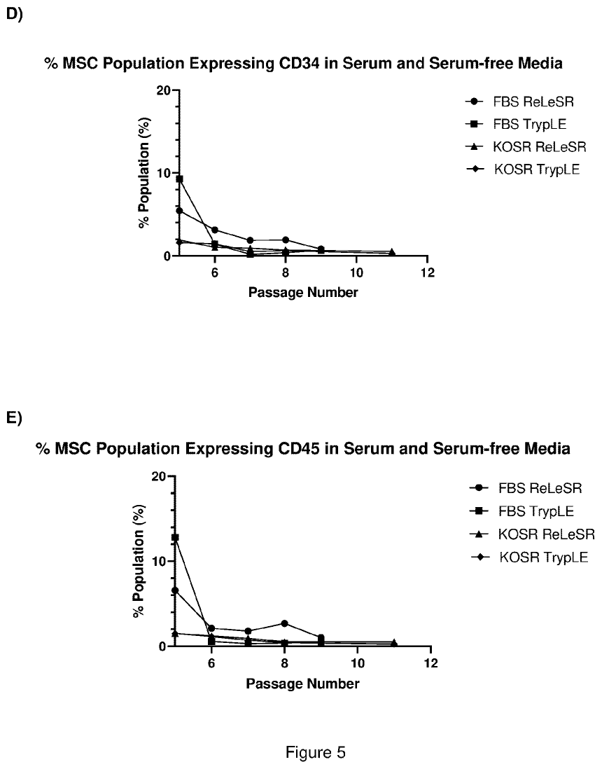

FIG. 5 shows flow cytometry analysis of MSC population that was generated with different methods. hiPSCs were dissociated with either TrypLE or ReLeSR, and the hiPSC-derived MSCs were generated using either FBS, or a serum-free defined alternative, KOSR. MSC populations used were cultured over the same period of time. A) shows the expression of CD73, B) shows the expression of CD90, C) shows the expression of CD105, D) shows the expression of CD34, E) shows the expression of CD45.

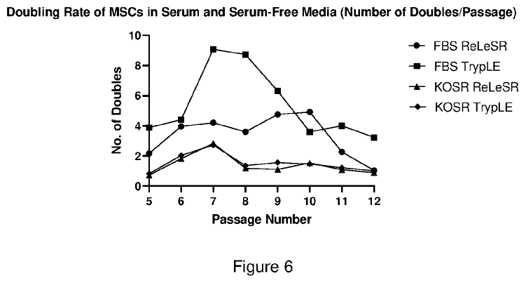

FIG. 6 shows the doubling rate of MSCs yielded over prolonged passaging. hiPSCs were dissociated with either TrypLE or ReLeSR, and the hiPSC-derived MSCs were generated using either FBS, or serum-free defined alternative, KOSR. MSC populations used were cultured over the same period of time.

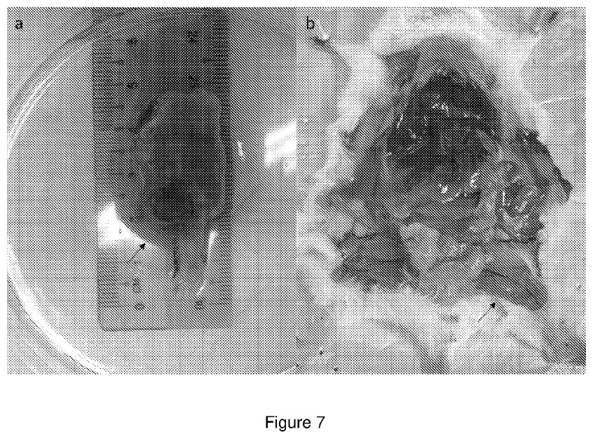

FIG. 7 shows representative autopsy images of nonobese diabetic severe combined immunodeficient (NOD SCID) mice injected with a) undifferentiated hiPSCs (positive control) and b) hPSC-derived MSCs generated with the serum-free protocol in accordance with the present embodiments, for teratoma assay. Arrows indicate site of injection.

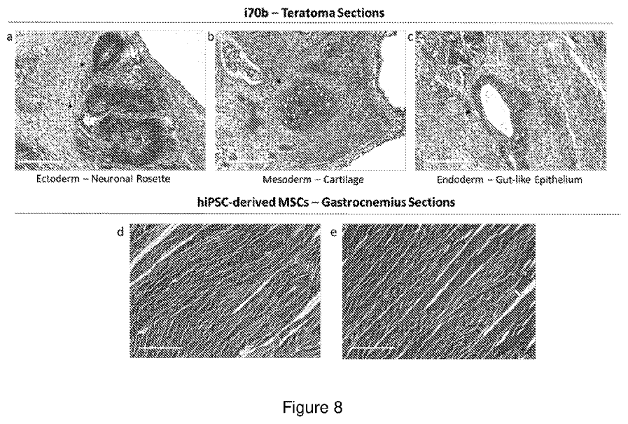

FIG. 8 shows histological analyses of the sites injected with (a, b, c) hPSCs, hPSC-derived MSCs generated with (d) serum-containing protocol and hPSC-derived MSCs generated with (e) serum-free protocol. Scale bar represents 100 μm and arrows indicate the respective germ layers.

hPSCs such as hiPSCs were differentiated into MSCs using serum-free media. An embodiment of the serum-free differentiation protocol is shown in FIG. 1. The culture media stated in each arrow in FIG. 1 are used to generate cells in the next process. Hence, MSC M1 (or MSC M0 and MSC M1 for TrypLE dissociated cells) are used to generate EBs from pluripotent stem cells. To summarise the protocol, pluripotent stem cells are first cultured in MSC M0 medium. After dissociation, clumps of cells (resulting from dissociation using ReLeSR) are cultured in MSC M1 for 8 days or, in the case of single cells (resulting from dissociation using TrypLE), cultured in MSC M0 for 1 day following by MSC M1 for the remaining 7 days to form and maintain the EBs. The EBs are typically formed 1-2 days in culture after the dissociation of pluripotent stem cells.

The hiPSCs were first dissociated into a suitable clump size or single cells for embryoid body formation. The dissociation step can be conducted using ReLeSR or TrypLE, which are both clinically-compatible reagents. With the use of ReLeSR, the hiPSCs are dissociated into clumps. With the use of TrypLE, the hiPSCs are dissociated into single cells. Subsequent pluripotent embryoid bodies were then replated and directed to differentiate into plastic-adherent MSCs. Various academic protocols were compared and modified (e.g. use of FBS, trypsin) such that the reagents used to generate hiPSC-derived MSCs were serum-free (e.g. use of KnockOut Serum Replacement (KOSR), TrypLE). These hiPSC-derived MSCs were characterised for their expression of MSC markers, which were found to be comparable with primary MSCs.

Existing protocols on serum-free donor-derived MSC cultures usually require the use of serum-containing reagents in the initial cultivation and early passages to maximise cell viability. Unlike conventional serum-free protocol, the method of the present invention allows for the use of serum-free reagents in the entire MSC cultivation pipeline, throughout all MSC passages, while maintaining the viability of MSCs.

The ability to differentiate hiPSCs into MSCs allows for the generation of unlimited quantities of MSCs from hiPSCs as opposed to the limited expansion potential of primary MSCs from human tissues. By creating a serum-free differentiation protocol, the generation of clinical-grade hiPSC-derived MSCs can be positioned for the eventual purpose of cell therapy and clinical applications in humans.

Existing FBS-containing protocols tend to be unreliable and highly variable due to batch-to-batch variability, differences in source and quality of FBS. In the present invention, existing FBS-containing differentiation protocols have been modified to be serum-free. This required the conscientious identification of serum-containing components to be swapped with serum-free reagents, and the verification that they can work equally well, if not better. FBS-containing protocols were compared with the serum-free protocol of the present invention and the cells were characterised (FIGS. 2 to 6). Advantageously, the protocol of the present invention is clinically compliant and is highly consistent in outcomes, making it suitable for use in biomanufacturing. In particular, the protocol of the present invention can be used to facilitate large-scale cell manufacturing processes.

The hPSC-derived MSCs generated by the method of the present invention can be used for various types of cell applications including chronic wounds, diabetic wounds, cardiovascular diseases, lung diseases (e.g. COVID-19), etc.

### Protocol for Serum-Free Differentiation

### Media Formulation

TABLE 1

| | | STAGE |
|---|---|---|
| | REAGENTS | APPLICABLE |
| HPSC M0 | DMEM(Dulbecco's Modified Eagle | hPSC Dissociation |
| MEDIUM | Medium)/F-12 with GlutaMAX | |
| | supplement | |
| | 20% KnockOut Serum Replacement | |
| | (KOSR) | |
| | 1% MEM non-essential amino acids | |
| | 1% Penicillin/Streptomycin | |
| MSC M0 | Essential 8 | EB Culture with |
| MEDIUM | 5-10 μM Y-27632 | Initial TrypLE |
| | | Dissociation |
| MSC M1 | DMEM (Dulbecco's Modified Eagle | EB Culture |
| MEDIUM | Medium) High Glucose | |
| | 15% KOSR | |
| | 1% GlutaMAX Supplement | |
| MSC M2 | DMEM High Glucose | Differentiation of |
| MEDIUM | 10% KOSR | EBs into MSCs |
| | 1% GlutaMAX Supplement | |
| MSC M3 | DMEM High Glucose | Maintenance of MSCs |
| MEDIUM | 15% KOSR | |
| | 1% GlutaMAX Supplement | |
| | 2.5 ng/mL Fibroblast Growth Factor | |
| | 2 (FGF-2) | |

In one embodiment, MSC M1 Medium consists of 74-89% DMEM, 10-25% KOSR and 1% GlutaMAX. In one embodiment, MSC M2 Medium consists of 79-89% DMEM, 10-20% KOSR and 1% GlutaMAX. In one embodiment, MSC M3 Medium consists of 74-84% DMEM, 15-25% KOSR, 1% GlutaMAX and 2.5-5 ng/ml FGF-2.

### Detailed Steps of Serum-Free Differentiation Protocol

### Day 1

Dissociate hPSCs with ReLeSR or TrypLE.
(a) For ReLeSR dissociation, add ReLeSR to the hPSCs at room temperature and remove ReLeSR within 30 seconds. Incubate hPSCs at 37° C. for 2.5-3.5 minutes for clump dissociation. Collect resultant hPSC aggregates with hPSC M0 medium and resuspend when necessary to yield hPSC clumps sizes between 50-200 μm. Seed hPSC clumps from a confluent 10 cm plate to a tissue culture non-treated six well plate with 3 mL of MSC M1 medium and 10 μM Rho-associated, coiled coil containing protein kinase (ROCK) inhibitor Y-27632 per well.
(b) For TrypLE dissociation, add TrypLE to the hPSCs and incubate at 37° C. for 3 minutes. Thereafter, aspirate TrypLE and collect hPSCs with hPSC M0 medium. Thoroughly resuspend hPSCs and filter through a 40 μM cell strainer to ensure a single cell suspension. Seed cells at 3-4 million cells/well in a tissue culture non-treated six well plate with 3 mL of MSC M0 medium and 10 μM ROCK inhibitor Y-27632 to prevent apoptosis.
Place the cell suspension on a shaker at 80-85 rpm for embryoid bodies (EBs) to form.

### Day 2

Change spent media with MSC M1 medium 24 h after cell seeding.
EBs should start to form.

### Day 4-7

Change spent media with MSC M1 medium every 2-3 days.

### Day 8

Replate EBs on a gelatin-coated six well plate with MSC M2 medium, with ˜20 EBs per well.

### Day 10-15

1) Change media with MSC M2 medium every 2-3 days.
2) When the well is confluent (˜5-7 days from replating), dissociate the cells with TrypLE and plate onto a gelatin-coated 10 cm plate with MSC M3 medium. This will be passage 1.
   Passage 1 onwards
1) Change media every 2-3 days with MSC M3 medium.
2) Cells might take longer to become confluent during the first passage and may require two weeks to grow before the next passage.
3) From passage 2 onwards, cells can be passaged on a seven-day cycle.
4) Cells can either be maintained on gelatin-coated plates or normal tissue culture-treated plates.
5) MSCs can be maintained with MSC M3 medium formulation for more than 15 passages, with >90% of the population expressing positive MSC markers (CD73, CD90 and CD105).

### Composition of Serum-Free Reagents

In one embodiment, different reagents were used in the serum-free formulation.

1) DMEM High Glucose (Catalogue Number SH30317 from Cytiva)

The composition of DMEM High Glucose is shown in Table 2.

TABLE 2

| | |
|---|---|
| COMPONENTS | MMOL/L |

INORGANIC SALTS

| | |
|---|---|
| CALCIUM CHLORIDE | 1.8021 |
| FERRIC NITRATE-9H20 | 0.0002 |
| POTASSIUM CHLORIDE | 5.3655 |
| MAGNESIUM SULFATE | 0.8112 |
| SODIUM CHLORIDE | 109.514 |
| SODIUM PHOSPHATE MONOBASIC H20 | 0.9059 |

AMINO ACIDS

| | |
|---|---|
| L-ARGININE-HCL | 0.3987 |
| L-CYSTINE-2HCL | 0.1998 |
| L-GLUTAMINE | 3.9959 |
| GLYCINE | 0.3996 |
| L-HISTIDINE-HCL-H20 | 0.2004 |
| L-ISOLEUCINE | 0.7989 |
| L-LEUCINE | 0.7990 |
| L-LYSINE-HCL | 0.8004 |
| L-METHIONINE | 0.2011 |
| L-PHENYLALANINE | 0.3995 |
| L-SERINE | 0.3997 |
| L-THREONINE | 0.7992 |
| L-TRYPTOPHAN | 0.0783 |
| L-TYROSINE-2NA-2H20 | 0.3974 |
| L-VALINE | 0.7990 |

VITAMINS

| | |
|---|---|
| CALCIUM D-PANTOTHENATE | 0.0084 |
| CHOLINE CHLORIDE | 0.0286 |
| FOLIC ACID | 0.0091 |
| MYO-INOSITOL | 0.0389 |
| NIACINAMIDE | 0.0328 |
| PYRIDOXINE-HCL | 0.0195 |
| RIBOFLAVIN | 0.0011 |
| THIAMINE-HCL | 0.0119 |

OTHER

| | |
|---|---|
| D-GLUCOSE | 24.9778 |
| PHENOL RED-NA | 0.0422 |
| SODIUM PYRUVATE | 0.9996 |
| SODIUM BICARBONATE | 44.0424 |

2) KOSR (Catalogue Number 10828028 from Cytiva and Thermo Fisher)

It is a defined, serum-free formulation. Unlike FBS, KOSR has a constant composition from one lot to another, thus eliminating the need to test every batch before being able to use it.

KOSR is composed of amino acids (glycine, L-histidine, L-isoleucine, L-methionine, L-phenylalanine, L-proline, L-hydroxyproline, L-serine, L-threonine, L-tryptophan, L-tyrosine, L-valine), vitamins/antioxidants (thiamine, reduced glutathione, ascorbic acid 2-PO₄), trace elements (Ag⁺, Al3⁺, Ba²⁺, Cd²⁺, Co²⁺, Cr³⁺, Ge⁴⁺, Se⁴⁺, Br⁻, I⁻, F⁻, Mn²⁺, Si⁴⁺, V⁵⁺, Mo⁶⁺, Ni²⁺, Rb⁺, Sn²⁺, Zr⁴⁺) and proteins (transferrin (iron-saturated), insulin, lipid-rich albumin (AlbuMAX)).

3) GlutaMAX Supplement (Catalogue Number 35050079 from Gibco or Thermo Fisher)

The composition of GlutaMAX Supplement is 200 mM L-alanyl-L-glutamine dipeptide in 0.85% NaCl.

4) FGF-2 (Catalogue Number 130-093-842 from Miltenyi Biotec)

The composition of the FGF-2 reagent is 100 ng/ml FGF-2 in sterile water.

5) TrypLE (Catalogue Number 12605010 from Thermo Fisher)

The composition of TrypLE is shown in Table 3.

TABLE 3

| | Concentration (mM) |
|---|---|
| Potassium Chloride | 2.6666667 |
| Potassium Phosphate monobasic | 1.4705882 |
| Sodium Chloride | 137.93103 |
| Sodium Phosphate dibasic | 8.059702 |
| EDTA | 1.1 |
| Phenol Red | 0.26567483 |
| rProtease | Proprietary |

6) ReLeSR (Catalogue Number 05872 from Stemcell Technologies)

This is a chemically defined, enzyme-free, animal component-free cGMP reagent for dissociation and passaging of hPSCs without manual selection or scraping. The drug master file is available from Stemcell Technologies.

7) DMEM/F-12 (Catalogue Number 10565018 from Thermo Fisher)

This is a 1:1 mixture of DMEM and Ham's F-12.

8 MEM Non-Essential Amino Acids (Catalogue Number 11140050 from Thermo Fisher)

This contains non-essential amino acids such as glycine, L-alanine, L-asparagine, L-aspartic acid, L-glutamic acid, L-proline and L-serine.

9) Penicillin/Streptomycin (Catalogue Number 15140122 from Thermo Fisher)

This solution contains 10,000 units/mL of penicillin and 10,000 μg/mL of streptomycin.

10) Essential 8 (Catalogue Number 05990 from Stemcell Technologies)

TeSR™-E8™ is a feeder-free, animal component-free culture medium for human embryonic stem cells and human induced pluripotent stem cells.

### Example 1

Human pluripotent stem cells (hPSCs) such as hiPSCs were differentiated into MSCs using serum-free media. Various academic protocols were compared and modified such that they were serum-free. For instance, FBS was replaced with KOSR and trypsin was replaced with TrypLE. These hiPSC-derived MSCs were characterised for their expression of MSC markers, which were shown in FIGS. 3 to 6 to be comparable with primary MSCs. The use of serum-free reagents to differentiate hiPSCs into MSCs positions these cells to be clinically-compliant that can eventually be used for downstream clinical applications.

### Example 2

As compared to the use of FBS, the method of the present invention was able to generate more EBs using the serum-free protocol by day 7. As seen in FIG. 4, EBs generated by the serum-free protocol of an embodiment of the present invention are more in numbers and consistent in size as compared to those generated with FBS-containing media. This difference is observable as early as on day 3 of the set up. The increase in the number of EBs, and consistency in size may be essential for initial scale-up efforts as lesser starting material will be required to yield a large amount of MSCs.

### Example 3

Cells cultured with the serum-free clinically-compliant method of the present invention have sustained MSC character over prolonged in vitro culture.

Based on the criteria laid out by the International Society for Cellular Therapy (ISCT), ≥95% of the MSC population must express CD73, CD90 and CD105, and ≤2% of the population can express negative MSC markers such as CD45, CD34, CD14 or CD11b, CD79a or CD19 and HLA class II.

Here, the relevant MSC positive (CD73, CD90, CD105) and negative markers expressed by MSCs generated with the serum-free differentiation protocol were compared with MSCs generated with the use of Fetal Bovine Serum (FBS).

FIGS. 5A-E show that the protocol of the present invention generates hiPSC-derived MSCs that consistently have ≥95% of the population expressing positive MSC markers (CD73, CD90, CD105) and ≤2% of the population expressing negative MSC markers (CD34 and CD45). In contrast, hiPSC-derived MSCs generated with the use of FBS exhibited a stark decrease in population expressing CD90 from passage 6 onwards. Percentage population expressing the negative MSC markers also fluctuates more in hiPSC-derived MSCs that are generated with FBS.

### Example 4

Cells generated using serum-free media exhibit more consistent doubling rate and can be passaged longer, without losing the ability to become confluent over a seven-day passage cycle.

As seen in FIG. 6, hiPSC-derived MSCs generated with the use of FBS have inconsistent doubling rate that may result in erratic passaging schedule due to fluctuating growth rates. In contrast, hiPSC-derived MSCs generated with the serum-free protocol of the present invention have consistent doubling rate (˜1× population doubling per passage) that can prevent batch-to-batch variation in growth rates.

### Example 5

To determine if there are any residual pluripotent stem cells in hPSC-derived MSCs generated with the serum-free protocol in accordance with the present embodiments, the hPSC-derived MSCs were injected into the gastrocnemius of immunodeficient NOD SCID mice and surveyed for teratoma formation (FIG. 7). In the positive control, where undifferentiated hPSCs were injected (FIG. 7a), teratoma formation was observed as early as eight weeks post-injection. However, no visible teratoma can be seen at the site of injection for NOD SCID mice that were transplanted with hPSC-derived MSCs for the same duration (FIG. 7b).

The in vivo teratoma assay demonstrated that the positive control undifferentiated hiPSC cell line, i70b, is pluripotent and can give rise to derivatives of the three germ layers corresponding to ectoderm (neuronal rosette), mesoderm (cartilage), and definitive endoderm (gut-like epithelium) (FIG. 8a, b, c). However, gastrocnemius tissue injected with hiPSC-derived MSCs only displayed normal striated muscle morphology (FIGS. 8d and e), suggesting that there were no more residual pluripotent stem cells in the hiPSC-derived MSCs. Hence, the serum-free differentiation protocol in accordance with the present embodiments is efficient in generating MSCs with no residual tumorigenic and undifferentiated hPSCs.

i70b was used as a starting hiPSC cell source for directed differentiation into MSCs. In vivo teratoma assay showed that i70b are pluripotent and can give rise to teratoma within eight weeks post-injection. In contrast, no visible tumors can be seen in the NOD SCID mice transplanted with hiPSC-derived MSCs generated with the serum-free protocol in accordance with the present embodiments. NOD SCID mice transplanted with hiPSC-derived MSCs remained tumor-free for 20 weeks and were eventually euthanized after 20 weeks in accordance with the IACUC protocol. Subsequent histological analysis of the tissue sites injected with the respective cell lines also verified that only undifferentiated i70b cells are pluripotent. Even after twenty weeks post-injection, gastrocnemius tissue injected with hiPSC-derived MSCs only contained normal mouse striated muscle cells, with no visible signs of teratoma cells. This suggests that the hPSC-derived MSCs generated using the serum-free protocol in accordance with the present embodiments are safe for transplantation.

In addition, directed differentiation of i70b into MSCs with conventional serum-containing protocol was also conducted based the protocol in Ahfeldt, T., et al., Programming human pluripotent stem cells into white and brown adipocytes. Nat Cell Biol, 2012. 14 (2): p. 209-19. Histological analysis showed that hiPSC-derived MSCs generated with the serum-free protocol in accordance with the present embodiments are comparable to those generated with the serum-containing protocol, with no residual pluripotent cells detectable through in vivo teratoma assay.

### Methodology for In Vivo Teratoma Assay

1×10⁶ i70b cells or i70b-derived MSCs were harvested for in vivo teratoma assay. The i70b-derived MSCs were cultured over six passages at the point of harvest. The cell pellet was resuspended in 100 to 150 μl of Matrigel. Then, the respective prepared cell lines were injected intramuscularly into the gastrocnemius of six-week-old NOD SCID mice. Mice injected with i70b developed teratoma within eight weeks post-injection and were euthanised. Meanwhile, no tumours could be detected by palpation or autopsy for mice injected with i70b-derived MSCs. These mice were only euthanised after twenty weeks. For further verification of teratoma formation, gastrocnemius of all mice was excised, fixed, paraffin sectioned and stained with Hematoxylin and Eosin (H&E) for subsequent analysis. All experiments on animals were performed in accordance with the relevant ethical regulations and approval from A*STAR with approval no. IACUC 211660.

While embodiments of the invention have been particularly shown and described with reference to specific embodiments, it should be understood by those skilled in the art that various changes in form and detail may be made therein without departing from the scope of the invention as defined by the appended claims. The scope of the invention is thus indicated by the appended claims and all changes which come within the meaning and range of equivalency of the claims are therefore intended to be embraced.

1. A method of differentiating pluripotent stem cells into multipotent mesenchymal stromal cells (MSCs), the method comprising the steps of:
(a) culturing the pluripotent stem cells in a first medium comprising Dulbecco's Modified Eagle Medium (DMEM), KnockOut™ Serum Replacement (KOSR) and GlutaMAX to form embryoid bodies (EBs);
(b) replacing the first medium with a second medium comprising DMEM, KOSR and GlutaMAX and culturing the EBs in the second medium to differentiate the EBs into MSC progenitors; and
(c) replacing the second medium with a third medium comprising DMEM, KOSR, GlutaMAX and Fibroblast Growth Factor 2 (FGF-2) and culturing the MSC progenitors in the third medium to differentiate the MSC progenitors into MSCs;
wherein the first medium, the second medium and the third medium are serum-free.

2. The method of claim 1, further comprising the step of dissociating the pluripotent stem cells into clumps using ReLeSR prior to step (a) or dissociating the pluripotent stem cells into single cells using TrypLE prior to step (a).

3. The method of claim 1, wherein the EBs and MSC progenitors are cultured on a gelatin-coated plate.

4. The method of claim 1, wherein the MSC progenitors are dissociated with TrypLE prior to step (c).

5. The method of claim 1, further comprising passaging the MSCs obtained in step (c) in the third medium to maintain the MSCs in a multipotent state.

6. The method of claim 5, wherein the MSCs are maintained in a multipotent state after 15 passages using the third medium.

7. The method of claim 5, wherein the MSCs are dissociated with TrypLE prior to each passage using the third medium.

8. The method of claim 1, wherein the pluripotent stem cells are human induced pluripotent stem cells (hiPSCs).

9. The method of claim 1, wherein after step (c), at least 95% of the population of cells obtained are MSCs.

10. A serum-free composition for differentiating pluripotent stem cells into multipotent mesenchymal stromal cells (MSCs), the composition comprising Dulbecco's Modified Eagle Medium (DMEM), KnockOut™ Serum Replacement (KOSR) and GlutaMAX.

11. The serum-free composition of claim 10, further comprising Fibroblast Growth Factor 2 (FGF-2).

12. The serum-free composition of claim 10, wherein DMEM is present at a concentration of about 74% to about 89% (v/v), KOSR is present at a concentration of about 10% to about 25% (v/v), and GlutaMAX is present at a concentration of about 1% (v/v).

13. The serum-free composition of claim 10, wherein DMEM is present at a concentration of about 79% to about 84% (v/v), KOSR is present at a concentration of about 15% to about 20% (v/v) and GlutaMAX is present at a concentration of about 1% (v/v).

14. The serum-free composition of claim 10, wherein DMEM is present at a concentration of about 79% to about 89% (v/v), KOSR is present at a concentration of about 10% to about 20% (v/v) and GlutaMAX is present at a concentration of about 1% (v/v).

15. The serum-free composition of claim 11, wherein DMEM is present at a concentration of about 74% to about 84% (v/v), KOSR is present at a concentration of about 15% to about 25% (v/v), GlutaMAX is present at a concentration of about 1% (v/v) and FGF-2 is present at a concentration of about 2.5 ng/ml to about 5 ng/mL.

16. The serum-free composition of claim 15, wherein FGF-2 is present at a concentration of about 2.5 ng/mL.

17. The serum-free composition of claim 10, wherein the pluripotent stem cells are human induced pluripotent stem cells (hiPSCs).

18. A population of MSCs obtainable by the method of claim 1.

19. The population of MSCs of claim 18, wherein at least 95% of cells in the population of MSCs express CD73, CD90 and CD105.

20. The population of MSCs of claim 18, wherein 2% or less of cells in the population of MSCs express CD34 and CD45.
